(19) United States
(12) Patent Application Publication
(10) Pub. No.: US 2016/0366855 A1
(43) Pub. Date: Dec. 22, 2016

(54) PET COMB APPARATUS

(71) Applicant: Jalyn Enterprise LLC, Lavon, TX (US)

(72) Inventor: Linda S. Jangula, Lavon, TX (US)

(21) Appl. No.: 15/183,466

(22) Filed: Jun. 15, 2016

Related U.S. Application Data
(60) Provisional application No. 62/180,694, filed on Jun. 17, 2015.

(51) Int. Cl. A01K 13/00; A61B 5/00

(52) U.S. Cl. CPC A01K 13/002; A01K 13/001; A61B 5/0013; A61B 5/0077; A61B 2503/40; H04W 84/12

(57) ABSTRACT

Disclosed is a pet comb apparatus. The pet comb apparatus includes an elongated shaft, an illumination system, and at least one row of a plurality of teeth extending outwardly and perpendicularly from the elongated shaft. The light from the illumination is used to locate fleas, ticks, insect eggs, skin infections and injuries on a pet.

### RELATED APPLICATIONS

Under the provisions of 35 U.S.C. §119(e), this application claims priority from provisional patent application No. 62/180,694, filed on Jun. 17, 2015 for a “Lighted Flea Comb For Pets” which is incorporated herein by reference in its entirety.

It is intended that each of the referenced applications may be applicable to the concepts and embodiments disclosed herein, even if such concepts and embodiments are disclosed in the referenced applications with different limitations and configurations and described using different examples and terminology.

### FIELD OF THE INVENTION

The invention generally relates to pet care appliances. More specifically, the invention relates to a pet hair comb.

### BACKGROUND

Pets, such as cats and dogs, need periodic grooming to retain their good health. Most pets play outdoors where dust and parasites get stuck on their bodies. In addition, as they have hairy body, they are vulnerable to insects such as fleas and ticks. However, it is important to address any flea or tick embedded within a pet's fur or hair in order to prevent an infestation or to prevent an infestation from spreading.

A comb is often used to loosen and remove fleas and ticks from the pet. Typical pet combs include a number of closely spaced teeth extending from a comb grip. When the comb is used, the teeth are able to loosen the fleas and ticks from the animal fur.

In order to detect an onset of an infestation, pet owners will carefully comb their pet in order to locate a flea, tick, or eggs thereof. However, it is extremely difficult to find a flea or tick on the pet due to their small size and the lack of proper illumination.

Therefore, there is a need for an improved comb for removal of fleas and ticks from a pet.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

Disclosed is a pet comb apparatus including an elongated shaft, an illumination system and one or more rows of a plurality of teeth extending outwardly and perpendicularly from the elongated shaft. The illumination system is configured to assist a pet owner to locate fleas, ticks, insect eggs, skin infections and injuries on a pet. The elongated shaft may be shaped to provide a comfortable grip and aesthetically appealing design. Further, the pet comb apparatus is made of one or more of plastic material, rubber and silicone.

In some embodiments, the elongated shaft includes a housing having an interior region. The illumination system resides in the interior region of the housing. In some alternate embodiments, a portion of the illumination system is situated in one or both of the shaft and at least one row of the plurality of teeth.

In some embodiments, the illumination system includes one or more light sources, a power source, and one or more control buttons that are operable to control the illumination system. The light sources are oriented to shine light generally along the direction of movement and away from the shaft. The one or more control buttons may be used to switch the illumination system between an on-state and off-state. Alternatively, the one or more control buttons may be used to control the intensity of light emitted by the one or more light sources.

In some further embodiments, the one or more light sources may emit high-energy rays to sterilize pet hair. For example, the high-energy rays may include ultraviolet rays or infrared rays.

In some embodiments, the elongated shaft includes an air heating apparatus and a blower configured for forcing out air heated by the air heating apparatus. The heated air is used to perform functions like drying wet hair and styling the hair.

In some embodiments, the pet comb apparatus includes a camera and a communication module. The camera is configured to take photos and videos. The communication module is configured to enable digital communication between the pet comb apparatus and an external device. The communication module may be further configured to provide one or both of wireless communication and wired communication, wherein the wireless communication includes at least one of Wi-Fi communication, Bluetooth communication and Zigbee communication. A pet owner may share photos and videos via the communication module with a remote veterinary doctor for remote diagnosis.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 is a perspective view a pet combing apparatus according to various embodiments.

FIG. 2 is a front view of the pet combing apparatus of FIG. 1.

FIG. 3 is a perspective view of the pet combing apparatus of FIG. 1.

FIG. 4 is a side view of the pet combing apparatus of FIG. 1.

FIG. 5 is a perspective view of the pet combing apparatus of FIG. 1.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of grooming pets, embodiments of the present disclosure are not limited to use only in this context.

The present disclosure relates to an illuminated pet comb, which includes an elongated shaft including a plurality of teeth that extend outwardly and perpendicularly therefrom. The teeth may be of a plurality different shapes and sizes. A pet owner holds the pet comb via the shaft; therefore, the shaft of the comb is shaped to provide a comfortable grip and aesthetically appealing design. The illuminated pet combing device further comprises a centrally located light assembly including a power source and a light source. During operation, the illuminated pet combing device is used to comb the fur or hair of the pet while the light is illuminated to allow the pet owner to locate fleas, ticks and eggs thereof on their pet.

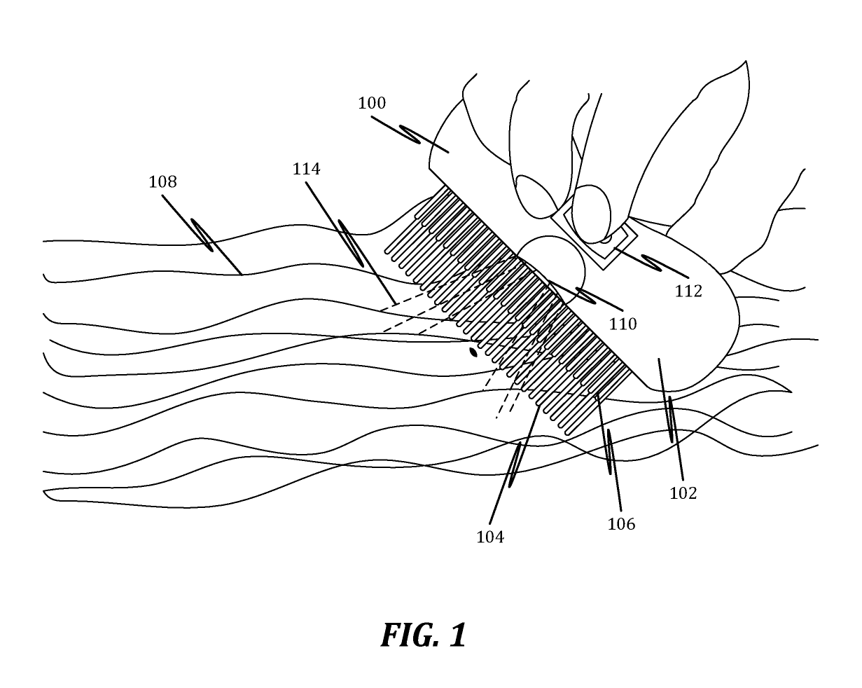

FIG. 1 illustrates a pet comb apparatus 100 according to some embodiments. The pet comb apparatus 100 includes an elongated shaft 102 and two rows 104-106 of multiple teeth extending outwardly and perpendicularly from the elongated shaft 102. In other embodiments, the pet comb apparatus 100 may have one or more rows of multiple teeth. The pet comb apparatus 100 is made of one or more of plastic material, rubber and silicone. The material of various parts of the pet comb apparatus 100 is selected based on the required stiffness and friction of the respective parts.

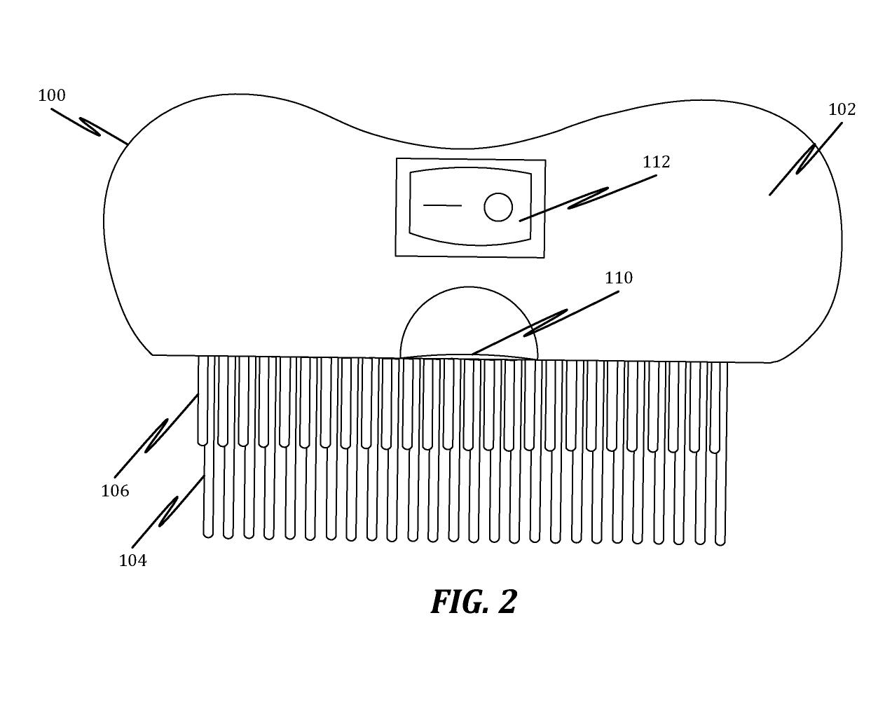

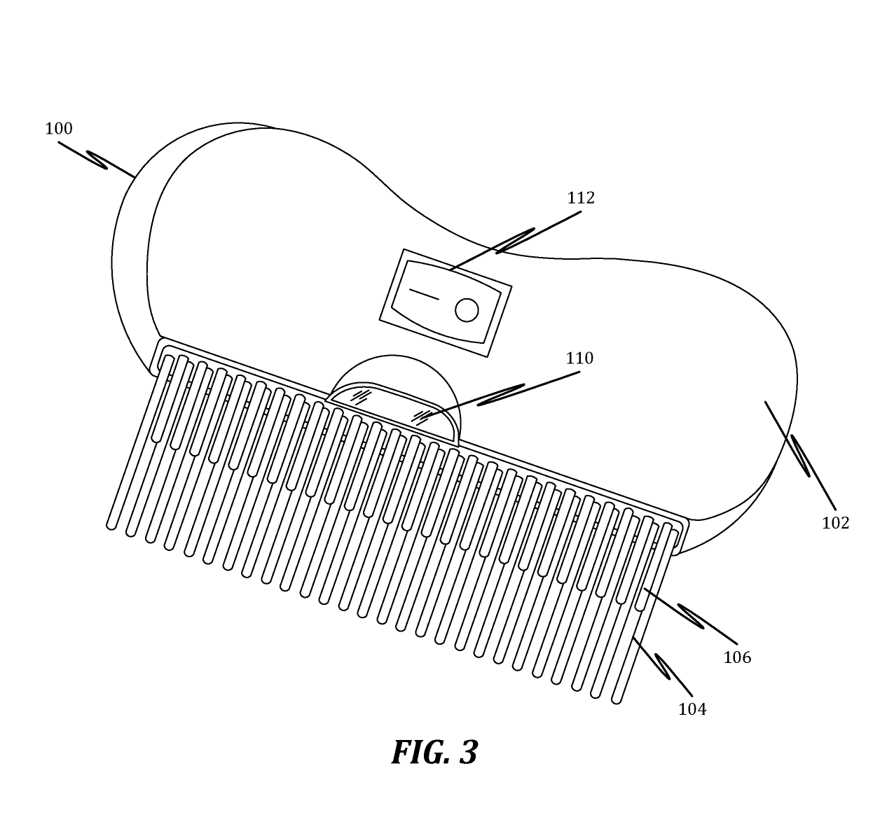

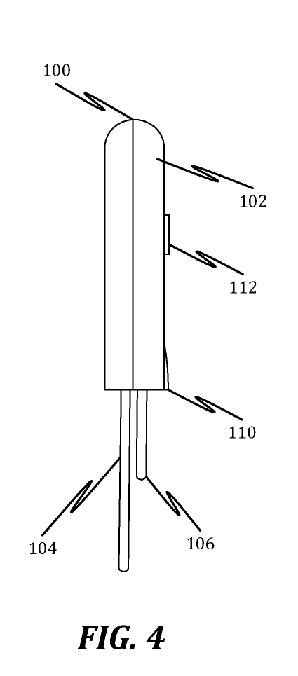

Referring now to FIGS. 2-4, showing a front view, a perspective view, and a side view, respectively, of the pet comb apparatus 100.

A pet owner may hold the pet comb apparatus 100 via the elongated shaft 102. The pet comb apparatus 100 may be used to comb the hair 108 of a pet as shown in FIG. 1. Accordingly, the elongated shaft 102 of the pet comb apparatus 100 may be shaped to provide a comfortable grip and aesthetically appealing design. For example, the shaft may be bone-shaped, rectangular shaped or may be given any other suitable shape.

The pet combing apparatus 100 further includes an illumination system, which may include one or more light sources 110, a power source, and one or more control buttons 112 that are operable to control the illumination system. The pet combing apparatus 100 further includes a control circuit that is operably connected to the one or more light sources, the power source, and the one or more control buttons 112. The power source may be rechargeable battery a replacement battery system or a non-replacement power source.

The elongated shaft 102 may include a housing having an interior region, wherein the illumination system resides in the housing. The one or more light sources 110 may be centrally located on the front surface of the elongated shaft 102. The one or more light sources 110 may include any suitable light source, such as light-emitting diodes (LEDs). Further, the one or more light sources 110 may be oriented such that when the illumination system is activated, the one or more light sources 110 may emit light 114 (as shown in FIG. 1) to shine generally along the direction of movement of the pet combing apparatus 100 and away from the housing of the pet combing apparatus 100. The light 114 allows a pet owner to locate one or more of fleas, ticks, insect eggs, skin infections and injuries on a pet.

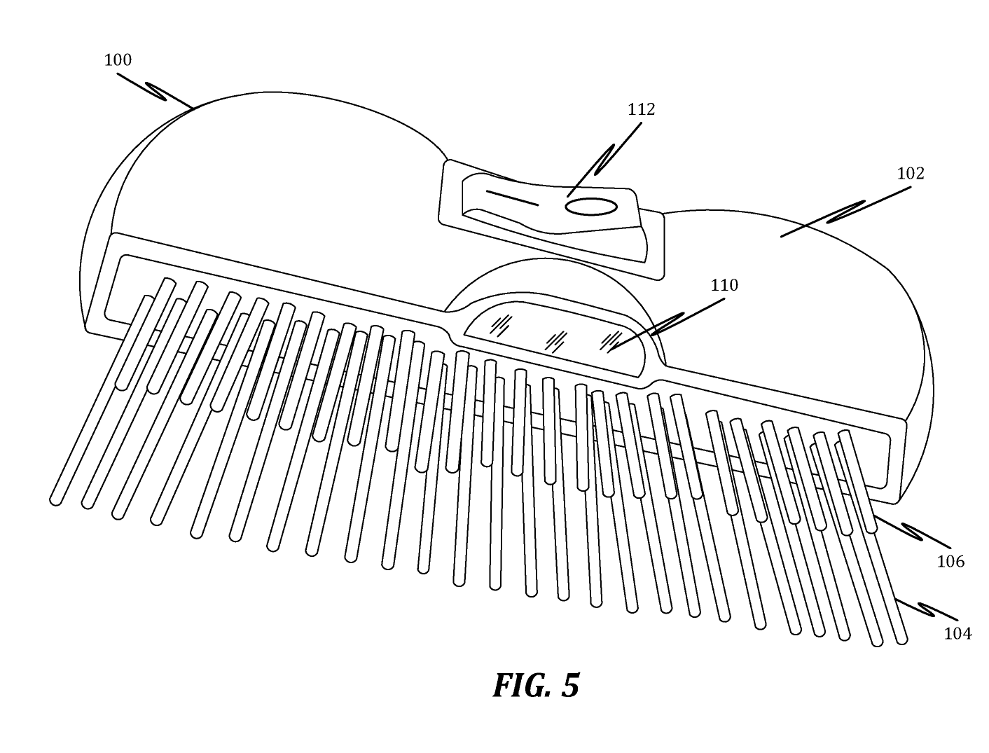

FIG. 5 shows a perspective view of the light 114 of the pet comb apparatus 100. The one or more control buttons 112 may allow switching the illumination system between an on-state and off-state; for example, the one or more control buttons 112 may include an on-off push button or any other style of on-off button. Alternatively, the one or more control buttons 112 may allow a user to control the intensity of light emitted by the one or more light sources 110; for example, the one or more control buttons 112 may include an adjustable switching regulator.

In some embodiments, at least one light source in the one or more light sources 110 emits high-energy radiation that may assist in sterilizing portions of a pet's hair. For example, the high-energy radiation may include infrared radiation and ultraviolet radiation.

In a further embodiment, the housing in the elongated shaft 102 may include an air heating apparatus, such as a convection heater. Further, the elongated shaft 102 may include a blower for forcing out the heated air from the housing towards the hair of a pet. The hot air may be used to dry wet hair or style the hair; for example, curl the hair.

In a yet further embodiment, the housing in the elongated shaft 102 may include a camera configured to take photos and videos of the area illuminated by the one or more light sources 110. The pet comb apparatus 100 may further include a communication module that allows a pet owner to retrieve the one or more photos from the pet comb apparatus 100. For example, the communication module may be configured to provide at least one of wired communication and wireless communication, such as Wi-Fi communication, Bluetooth communication or Zigbee communication. The pet owner may then share the photos or the videos, via the communication module, with a remote veterinary doctor for remote diagnosis.

In an alternate embodiment, at least some of the teeth in the two rows 104-106 of multiple teeth are hollow. Further, at least some light sources in the one or more light sources 110 are arranged such that the light emitted by the at least some light sources travels via the hollow teeth towards the hair of the pet. In a further embodiment, at least a portion of the hollow teeth is transparent, such that light traveling through the hollow teeth disperses out of the transparent portions to provide more illumination over the pet hair.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A pet comb apparatus comprising:
an elongated shaft;
an illumination system; and
at least one row of a plurality of teeth extending outwardly and perpendicularly from the elongated shaft.

2. The pet comb apparatus of claim 1, wherein the elongated shaft is of a shape adapted to provide at least one of a comfortable grip and aesthetically appealing design.

3. The pet comb apparatus of claim 1, wherein the pet comb apparatus is made of at least one of plastic material, rubber and silicone.

4. The pet comb apparatus of claim 1, wherein at least a portion of the illumination system is situated in at least one of the elongated shaft and the at least one row of the plurality of teeth.

5. The pet comb apparatus of claim 1, wherein the elongated shaft comprises a housing having an interior region, wherein the illumination system resides in the interior region of the housing.

6. The pet comb apparatus of claim 5, wherein the illumination system comprises at least one light source, a power source, and at least one control button that is operable to control the illumination system.

7. The pet comb of claim 6, wherein the at least one light source is oriented to shine light generally along the direction of movement and away from the housing.

8. The pet comb apparatus of claim 7, wherein the light emitted by the at least one light source is used to locate at least one of fleas, ticks, insect eggs, skin infections and injuries on a pet.

9. The pet comb apparatus of claim 6, wherein the at least one light source emits high-energy rays to sterilize pet hair.

10. The pet comb apparatus of claim 9, wherein the high-energy rays are at least one of ultraviolet rays and infrared rays.

11. The pet comb apparatus of claim 6, wherein the at least one control button is used to switch the illumination system between an on-state and off-state.

12. The pet comb apparatus of claim 6, wherein the at least one control button is used to control the intensity of light emitted by the at least one light source.

13. The pet comb apparatus of claim 5, wherein the elongated shaft comprises an air heating apparatus and a blower configured for forcing out the air heated by the air heating apparatus.

14. The pet comb apparatus of claim 13, wherein the heated air is used to perform at least one of drying wet hair and styling the hair.

15. The pet comb apparatus of claim 1 further comprising a camera and a communication module, wherein the camera is configured to take at least one photo and the communication module is configured to enable digital communication between the pet comb apparatus and an external device.

16. The pet comb apparatus of claim 15, wherein the communication module is configured to provide at least one of a wireless communication and a wired communication.

17. The pet comb apparatus of claim 16, wherein the wireless communication comprises at least one of Wi-Fi communication, Bluetooth communication and Zigbee communication.

18. The pet comb apparatus of claim 15, wherein the at least one photo is shared with a remote veterinary doctor, via the communication module, for remote diagnosis.

19. The pet comb apparatus of claim 1, wherein at least one teeth in the plurality of teeth is hollow, wherein the at least one light source is arranged such that the light emitted by the at least one light source travels via the hollow teeth towards the hair of the pet.

20. The pet comb apparatus of claim 1, wherein at least a portion of the hollow teeth is transparent, such that light traveling through the hollow teeth disperses out of the transparent portion to provide more illumination over the hair of the pet.
